(19) United States
(12) Patent Application Publication
(10) Pub. No.: US 2020/0121258 A1
(43) Pub. Date: Apr. 23, 2020

(54) WEARABLE DEVICE FOR NON-INVASIVE ADMINISTRATION OF CONTINUOUS BLOOD PRESSURE MONITORING WITHOUT CUFFING

(71) Applicant: AlayaTec, Inc., Palo Alto, CA (US)

(72) Inventors: Li Zhu, Cupertino, CA (US); David H.C. Chen, Palo Alto, CA (US)

(21) Appl. No.: 16/164,777

(22) Filed: Oct. 18, 2018

(51) Int. Cl. A61B 5/00; A61B 5/021; A61B 5/026; A61B 5/044; A61B 5/0295

(52) U.S. Cl. CPC A61B 5/7278; A61B 5/02125; A61B 5/0261; A61B 5/6804; A61B 5/0022; A61B 2562/164; A61B 5/044; A61B 5/746; A61B 5/0295; A61B 5/681; A61B 2503/40; A61B 5/7267

(57) ABSTRACT

A wearable blood pressure monitoring device includes a housing with a processor and array of sensors is suitable to continuously wear on a subject without cuffing the subject during measurements. The sensors include an ECG sensor and a PPG sensor in contact with the external surface of the skin of the subject. The processor determines blood pressure from a determined PTT value resulting from a time difference between the measured ECG signal and the measured PPG signal resulting from the heartbeat.

### FIELD OF THE INVENTION

The present invention relates generally to a wearable blood pressure monitoring device, and more specifically to, a wearable blood pressure monitoring device for non-invasive administration of continuous and cuffless blood pressure readings for cardiac activity of a subject.

### BACKGROUND

Many patients have their blood pressure taken one or many times during a hospital visit. Typically, an instrument called a sphygmomanometer with a cuff is placed around a patient's arm and inflated with a pump until circulation is cut off. A small valve slowly deflates the cuff, and a health professional uses a stethoscope placed over an arm to listen for the sound of blood pulsing through the arteries. Outside of the hospital, blood pressure can be taken at a pharmacy or even at home for some. Problematically, these intermittent readings consume time and can inconveniently require removal of garments.

When blood pressure moves out of range, this can be a warning sign for heart attacks, hypertension, general health, and other issues. More generally, the heart has two upper chambers for entry of blood and two lower chambers for contracting to send blood through circulation. The cardiac cycle refers to a complete heartbeat from its generation to the beginning of the next heartbeat. The heart operates automatically to rhythmically contract. Blood pressure is related to the force and rate of each heartbeat and the diameter and elasticity of arterial walls. Systolic blood pressure (SBP) indicates how much pressure blood is exerting against artery walls during heart beats. Diastolic blood pressure (DBP) indicates how much pressure blood is exerting against artery walls while the heart is resting between beats. Blood pressure can be measured in millimeters of mercury or mm Hg.

Conventional techniques for taking blood pressure require cuffing and others are invasive. Cuffing refers to wrapping a sleeve from a blood pressure device around an arm and pressurizing the sleeve. One invasive manner of blood pressure measurement is from intra-arterial sheath inserted in the body. Many of these conventional solutions are not practical for monitoring patients over longer periods of time. As a result, intermittent readings are taken, and patients can be at risk in between blood pressure readings. Otherwise, patients remain permanently tethered to a machine for continuous readings, losing mobility.

What is desired is a technique for wearable blood pressure monitoring device for non-invasive administration of continuous and cuffless blood pressure readings for cardiac activity of a subject.

### SUMMARY

The above-described shortcomings are resolved by a system, method, and source code associated with a wearable blood pressure monitoring device for administering continuous and cuffless blood pressure readings of cardiac activity of a human subject.

In one embodiment, a wearable blood pressure monitoring device includes a housing is suitable to continuously wear on a subject without cuffing the subject during measurements. A processor is embedded within the housing. An array of physiological sensors in electrical communication with the processor are attached to the housing.

In another embodiment, the sensors include an Electrocardiogram (ECG or EKG) sensor and a Photoplethysmogram (PPG) sensor in contact with the external surface of the skin of the subject. The ECG sensor has electrodes to periodically measure electrical potential from a heartbeat from the electrodes at different locations on the skin of the subject. The PPG sensor periodically measures blood volume changes resulted from the heartbeat from at least one location of the skin of the subject. The processor determines blood pressure from a determined Pulse Transit Time (PTT) value resulting from a time difference in, for example, peak values, 50% of rising edge, or valley values, between the measured ECG signal and the measured PPG signal.

In still another embodiment, an output of the wearable blood pressure monitoring device automatically notifies as preconfigured, responsive to blood pressure falling outside of a predetermined range for the heartbeat.

Advantageously, patient blood pressure levels can be continually monitored in a manner that is comfortable for everyday activities, non-invasive, and cuffless.

### BRIEF DESCRIPTION OF THE DRAWINGS

In the following drawings, like reference numbers are used to refer to like elements. Although the following figures depict various examples of the invention, the invention is not limited to the examples depicted in the figures.

FIG. 1 is a high-level block diagram illustrating a system for a wearable blood pressure monitoring device for non-invasive administration of continuous and cuffless blood pressure readings for cardiac activity of a subject, according to an embodiment.

FIG. 2 is an exemplary block diagram illustrating a wearable blood pressure device for blood pressure monitoring for non-invasive administration of continuous and cuffless blood pressure readings for cardiac activity of a subject, according to an embodiment.

FIG. 3 is a more detailed exemplary block diagram illustrating a flexible motherboard as a substrate for components of the wearable blood pressure device of FIG. 2, according to an embodiment.

FIGS. 4-6 are high-level exemplary flow charts illustrating various methods for wearable blood pressure monitoring device for non-invasive administration of continuous and cuffless blood pressure readings for cardiac activity of a subject, according to an embodiment.

### DETAILED DESCRIPTION

The following description presents systems, methods, and source code (e.g., non-transitory source code stored on a computer-readable medium for execution by a processor) for a wearable blood pressure monitoring device for non-invasive administration of continuous and cuffless blood pressure readings for cardiac activity of a human subject.

This description is intended to enable one of ordinary skill in the art to make and use the embodiments and is provided in the context of a patent application and its requirements. Various modifications to the preferred embodiments and the generic principles and features described herein will be readily apparent to those skilled in the art. For example, a wearable blood pressure device can be adapted for use on non-humans (e.g., dogs) or for other biometric data. Thus, the present embodiments are not intended to be limited as shown, but are to be accorded the widest scope consistent with the principles and features described herein.

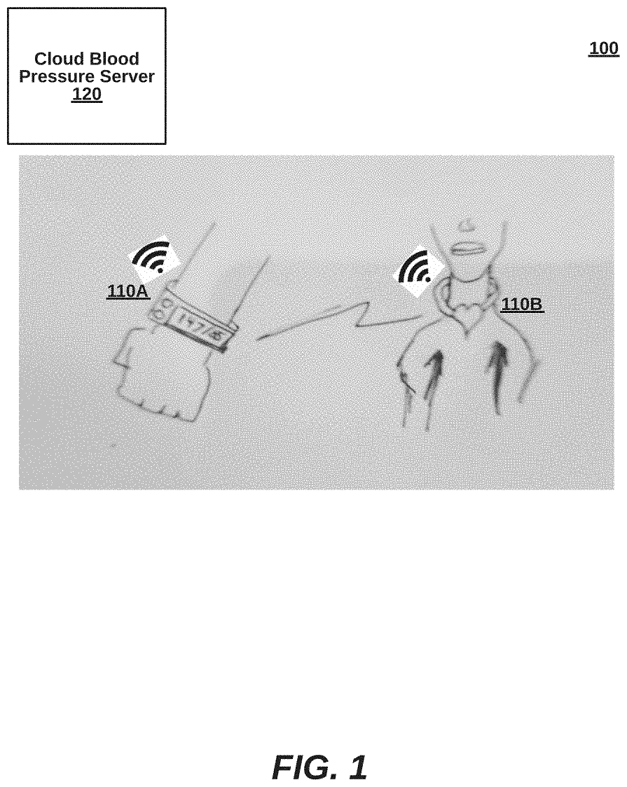

I. Device Administering Continuous and Cuffless Blood Pressure Readings for Cardiac Activity of a Subject (FIGS. 1-3)

FIG. 1 is a perspective diagram illustrating wearable blood pressure monitoring system 100 for non-invasive administration of continuous and cuffless blood pressure readings for cardiac activity of a subject, according to an embodiment. The system includes various types of blood pressure devices 110A, 110B worn on the wrist or neck and a cloud blood pressure server 120. The blood pressure monitoring devices 110A, 110B can be implemented as, for example, a ring, a wrist band/bracelet, a watch, an arm band, a necklace, a headset, an earbud, a belt, a waist band, a patch, a garment, a shoe accessory, an ankle band, or any combination thereof, or the like. Because blood pressure devices are wearable, cuffless and non-invasive, they can be taken continuously while in a hospital environment, or taken during a wide range of activities outside of the hospital environment. The blood pressure monitoring devices 110A, 110B in some embodiments are independently-operating devices, and in other embodiments, are cooperating devices (e.g., two different types of sensors, a central device and remote electrode).

In one example, a patient is able to monitor blood pressure throughout everyday activities and is notified when out of a safe range. In another example, a wet suit for scuba diving has integrated blood pressure monitoring for divers during deep dives which can cause a change in blood pressure to a range abnormal for the diver. In still another example, a jogger or other athlete is able to adjust a running pace to stay within a safe blood pressure range.

In another embodiment, smart textiles or e-textiles, utilizing wearable garments as the connecting platform for distributed sensors and other electronic components, such as provided by IMEC (Interuniversitair Micro-Electronica Centrum) of The Netherlands. The processor, the memory element, and the output can each be affixed or laminated to the wearable garment preferably during the material production processes or when stitched, for full integration. Some components, however, can be accessible for switching out or upgrading. A micro USB or other generic connector or wired or wireless port can provide an open system for connecting new hardware. The wearable garment can be made from fabric and comprise a shirt, a pants, a shoe, a hat, a glove, underwear, a sock, a band, a tape or any other type of appropriate wearable garment.

Further, components can have wireless capability for wireless communication. The cloud blood pressure server 120 supports remote processing with a large database, artificial intelligence (AI) capabilities, and more processing power for offloading and/or uploading. A wireless connection upstream can be enabled by other network components, such as access points, smart phones with Wi-Fi or cellular connections, Bluetooth transceivers, and the like. The cloud blood pressure server 120 can provide software as a service to many users with secure user accounts. A physician or hospital sever can get push or pull updates to track a specific patient. AI processes can use historical data from users over time to generate statistical models and to train other components.

Output mechanisms for the wearable blood pressure device can automatically notify the patient and others. A bright light, buzzer, or vibration can alert a patient. If network capable, an emergency dispatch service, doctor, caregiver, relative or friend can be notified electronically via SMS, e-mail or the like.

The blood pressure monitoring devices 110A, 110B can be a standalone device merely for blood pressure monitoring, or a combination device used also for monitoring other vital signs. Heart rate, Inter Beat Intervals (IBIs), and Heart Rate Variability (HRV) can also be determined. Many other embodiments are possible, given the disclosure herein, although not described in detail in the interest of brevity.

The cloud blood pressure server 120 supports remote processing with a large database, AI capabilities, and more processing power for offloading and/or uploading. A wireless connection upstream can be enabled by other network components, such as access points, smart phones with Wi-Fi or cellular connections, Bluetooth transceivers, and the like. The cloud blood pressure server 120 can provide software as a service to many users with secure user accounts. A physician or hospital server can get push or pull updates to track a specific patient. AI processes can use historical data from users over time to generate statistical models and to train other components.

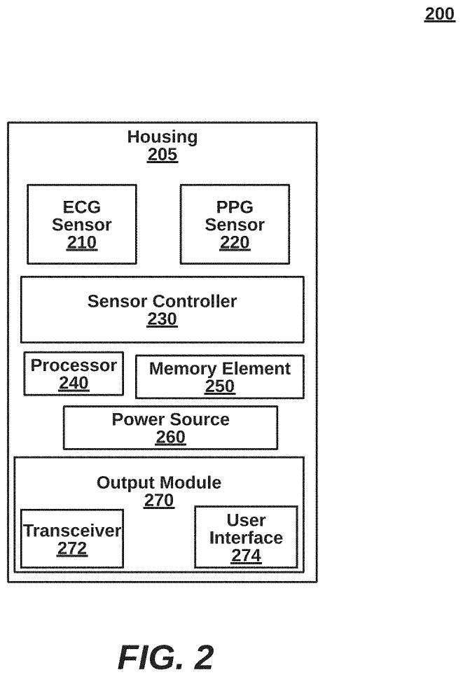

FIG. 2 is an exemplary block diagram illustrating an embodiment of a blood pressure device 200 for wearable blood pressure monitoring device for non-invasive administration of continuous and cuffless blood pressure readings for cardiac activity of a subject, according to an embodiment. The blood pressure device 200 comprises a housing 205, an ECG sensor 210, a PPG sensor 220, a sensor controller 230, a processor 240, a memory element 250, a power supply 260, and an I/O module 270. The components can be implemented in hardware, software, or any combination thereof.

The housing 205 can be driven by the type of wearable device (e.g., watch versus ring) and ornamental designs, in addition to functionally protecting electronic components. Straps can be attached to the housing for attachment to a wrist, waist, neck, or ankle of a subject, for instance. The housing 205 can be made from one or more of any appropriate materials such as plastic, rubber, metal, leather and glass. Some components of the blood pressure device 200, such as electrodes, can be located remote from the housing 205 and be connected by radio or conductive wiring.

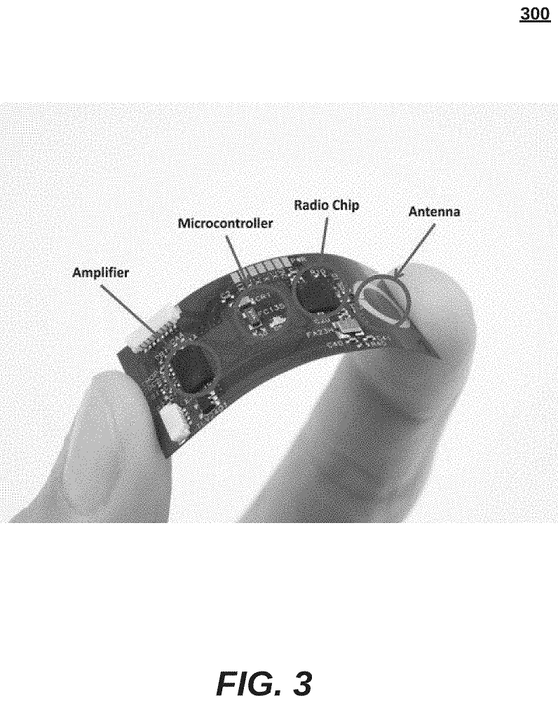

In one embodiment, a flexible motherboard 300 provides a wearable substrate for the components, as shown in FIG. 3. The processor, the memory element, the sensors, the transceiver, and the output can each be affixed to the motherboard 300 during manufacturing. The motherboard 300 can be, for example, a flexible wireless ECG sensor with fully functional microcontroller, by IMEC of the Netherlands. Alternative embodiment use standard, rigid motherboards.

Returning to the blood pressure device 200 of FIG. 2, the ECG sensor 210 includes two or more electrodes to sense electric activity of the heart at different locations, as a first form of cuffless and non-invasive blood pressure measurement. The electrodes can be wirelessly connected to the housing or be connected with a wire. More generally, ECG records electrical activity generated by heart muscle depolarizations, which propagate in pulsating electrical waves towards the skin. The electrodes are in contact with the skin and pick up very small amounts of electricity in microvolts (pV). In one example, electrodes are in contact at one or more of a right arm, a left arm, a right leg and a left leg. Conductive gel or electrode covers can be used on the electrodes to increase conductivity with the skin.

The PPG sensor 220 detects volume change caused by a blood pressure pulse, as a second form of cuffless and non-invasive blood pressure measurement. In more details, throughout the cardiac cycle, blood pressure around the body increases and decreases, even in the outer layers and small vessels of the skin. Peripheral blood flow can be measured using optical sensors in contact with the fingertip, the ear lobe or other capillary tissue, for example. One or more light emitting diode (LED), low power laser, or other light source can send light into the tissue and record how much light is either absorbed or reflected to a photodiode or other light sensor.

In a first embodiment, the sensor control module 230 utilizes the ECG module 210 to activate electrical readings and the PPG module 220 to activate blood volume readings. The processor 240 calculates criteria needed to determine blood pressure. In one case, the processor 240 considers the time it takes for a pulse wave to travel between two arterial locations (PTT). A linear or nonlinear model is then built between PTT and SBP/DBP values via machine learning or deep learning algorithm (e.g., Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks, any appropriate combination or the like). In some devices, an analog-to-digital converter (ADC) works along with the sensor control module 230 as part of a general control system, such as a smart watch that monitors many activities and performs other smart watch functions. In thin devices, the sensor control module 230 can be the main controller without the need for a processor 240 because larger processing tasks are offloaded or uploaded to a cloud service.

In another case, Recurrent Neural Networks (RNN), Long Short Time Memory Networks (LSTM), other algorithms, or any combination thereof are implemented by considering the synchronized ECG/PPG as multivariate time series, which are mapped to the SBP and DBP values directly.

In still another case, the ECG/PPG time series are firstly converted into multilayer graphs, based on an algorithm such that the spatial characteristics of the graphs inherit the temporal characteristics of the ECG/PPG time series. The algorithm may be Visibility Graph (VG) or other algorithm. A Neural Networks or other machine learning or deep learning algorithm is then implemented to map the multilayer graphs to the SBP and DBP values.

In a second embodiment, the PTT is firstly extracted from the multiple PPG signals by the processor 240. A linear or nonlinear model is then built between PTT and SBP/DBP values via machine learning or deep learning algorithms (e.g., Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks, any combination thereof, or the like).

Alternatively, a RNN, LSTM, or other algorithms are implemented by considering the synchronized PPG signals as multivariate time series, which are mapped to the SBP and DBP values directly.

Additionally, the PPG time series are firstly converted into multilayer graphs, based on an algorithm such that the spatial characteristics of the graphs inherit the temporal characteristics of the PPG time series. The algorithm may be VG or other algorithm or statistical analysis. A Neural Networks or other machine learning or deep learning algorithm is then implemented to map the multilayer graphs to the SBP and DBP values.

In a third embodiment, RNN, LSTM, or other algorithms are implemented based on a single PPG signal by considering the PPG signal as time series, which are mapped to the SBP and DBP values directly.

In another alternative, the single PPG signal is firstly converted into a graph, based on an algorithm such that the spatial characteristics of the graph inherit the temporal characteristics of the PPG signal. The algorithm can be VG or the like. A Neural Networks or other machine learning or deep learning algorithm is then implemented to map the graphs to the SBP and DBP values.

An implementation may present the estimated SBP and DBP values to the user via a display, or via earbuds using voice or sounds. The results may also be transmitted to a healthcare provider via a network. The DSP and/or cloud server may optionally execute a second computational model to estimate the heart rate (HR) from ECG and/or PPG signals simultaneously.

The processor 240 can be a microcontroller unit (MCU), application processor (AP), central processing unit (CPU), floating point unit (FPU), digital signal processor (DSP), system on a chip (SoC), other computational hardware, or a combination thereof. An embodiment may deploy STM32 from STMicroelectronics, or similar commercial products, as a microcontroller unit (MCU). The processor 240 can be single core, multiple core, or include more than one processing elements. The processor 240 can be disposed on silicon or any other suitable material. The processor 240 can receive and execute instructions and data stored in the cache or the memory element 250.

The memory element 250 of the blood pressure device 200 can be any non-volatile type of storage such as a magnetic disc, EEPROM, Flash, or the like. Memory element 250 stores code and data for applications.

The power supply 260 can be a one-time battery or a rechargeable battery. A USB port or other wired or wireless connector can provide power for recharging. In other cases, a standard one time watch battery can also power blood pressure measuring electronics. In still other cases, the power supply 260 can be a solar energy system or the like. The solar energy system involves a solar energy panel, a battery, and a charge controller, and accessories. Alternatively, the power supply 260 can be a connector to the power outlet.

The transceiver 270 connects to a medium such as Ethernet or Wi-Fi, Bluetooth, Zigbee, near-field communication (NFC), or the like for data input and output. In one embodiment, the network interface includes IEEE 802.11 antennae.

The output module 270 can include a transceiver 272 and a user interface 274, and can be a one or more of a display, an LED, a speaker, an interface to electronic notifications such as e-mail or short message service (SMS), a vibration element, and the like. An LED light can flash to notify a human subject wearing the blood pressure device 200. A remote physician or hospital server can be notified of blood pressure data, and notifications can be initiated from the remote physician or hospital server. In another case, blood pressure data is sent privately to an AI server performing analytics on different sets of data. Some implementations include just the transceiver 272 or just the user interface 274.

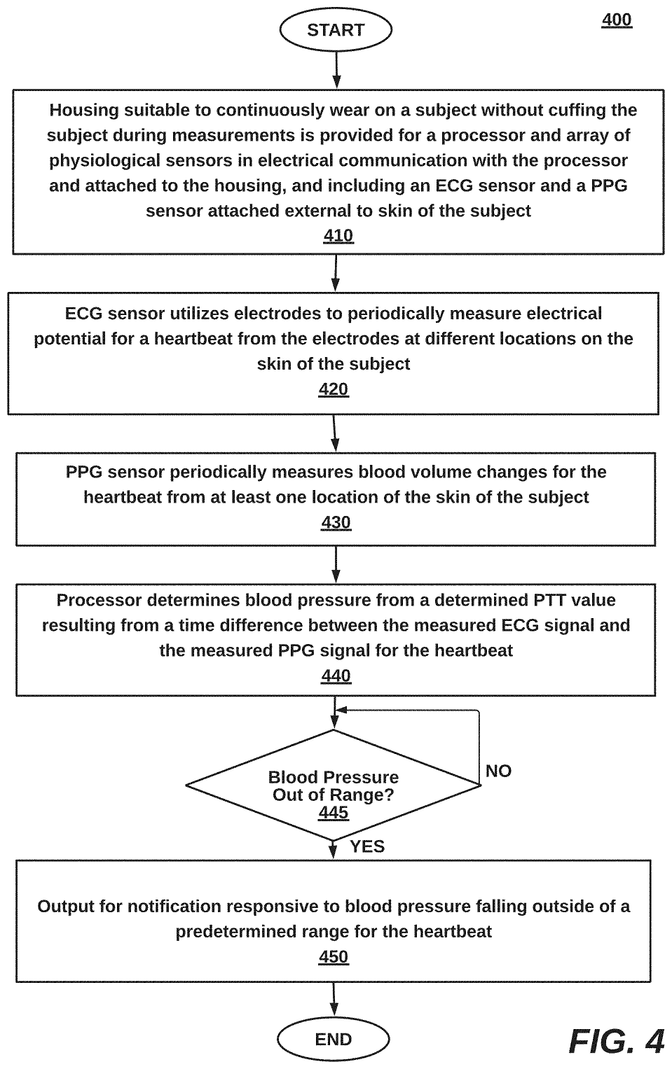

II. Methods Administering Continuous and Cuffless Blood Pressure Readings for Cardiac Activity of a Subject (FIG. 4-6)

FIGS. 4-6 are flow charts illustrating a method 400 for wearable blood pressure monitoring device for non-invasive administration of continuous and cuffless blood pressure readings for cardiac activity of a subject, according to an embodiment. The methods 400-600 can be implemented in devices 100A,B of FIG. 1 or others. The steps are generally groupings of functionality and can be performed in a different order, or in parallel, with additional steps and sub-steps.

At step 410 a housing suitable to continuously wear on a subject without cuffing the subject during measurements is provided for a processor and array of physiological sensors in electrical communication with the processor and attached to the housing, and including an ECG sensor and a PPG sensor in contact with the external surface of the skin of the subject.

At step 420 the ECG sensor utilizes electrodes to periodically measure electrical potential for a heartbeat from the electrodes at different locations on the skin of the subject.

At step 430, the PPG sensor periodically measures blood volume changes resulting from the heartbeat from at least one location of the skin of the subject.

At step 440, the processor determines blood pressure by utilizing the temporal information from the measured ECG signal and the measured PPG signal resulted from the heartbeat.

At step 445, the processor determines if the determined blood pressure is outside of a predetermined range for the heartbeat. Other embodiments perform or do not perform step 445.

At step 450, an output for notification responsive to blood pressure falling outside of the predetermined range.

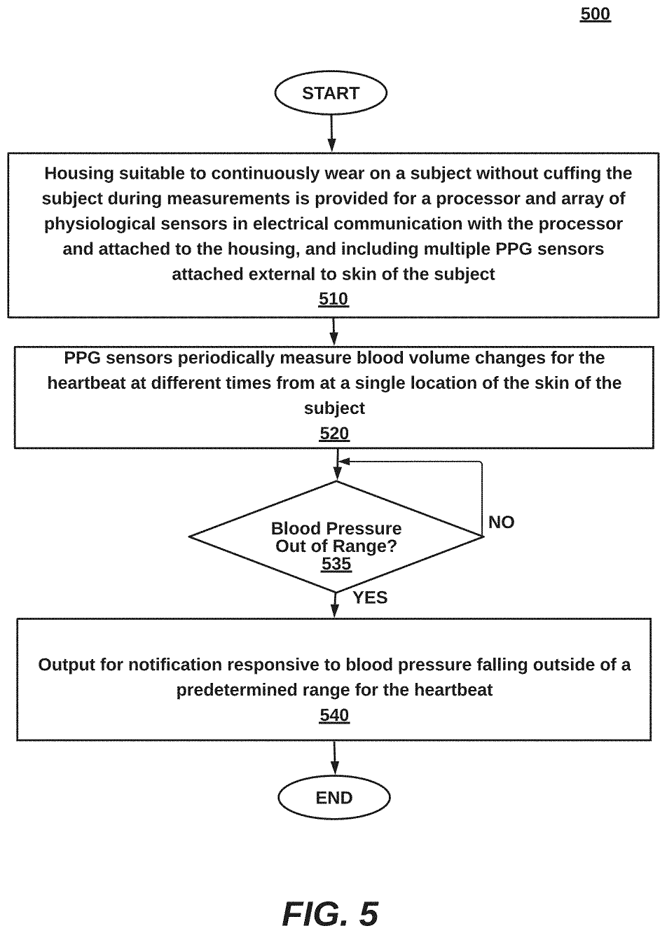

FIG. 5 is a flow chart illustrating a method 500 for wearable blood pressure monitoring device for non-invasive measurement of continuous and cuffless blood pressure readings with multiple PPG sensors.

At step 510 a housing suitable to continuously wear on a subject without cuffing the subject during measurements is provided for a processor and array of physiological sensors in electrical communication with the processor and attached to the housing, and including multiple PPG sensors in contact with the external surface of the skin of the subject.

At step 520, the PPG sensors periodically measure blood volume changes resulted from the heartbeat from different locations of the skin of the subject.

At step 530, the processor determines blood pressure by utilizing the temporal information from the measured PPG signals at different locations resulting from the heartbeat.

At step 535, the processor determines if the determined blood pressure is outside of a predetermined range for the heartbeat. Other embodiments perform or do not perform step 535.

At step 540, an output for notification responsive to blood pressure falling outside of the predetermined range.

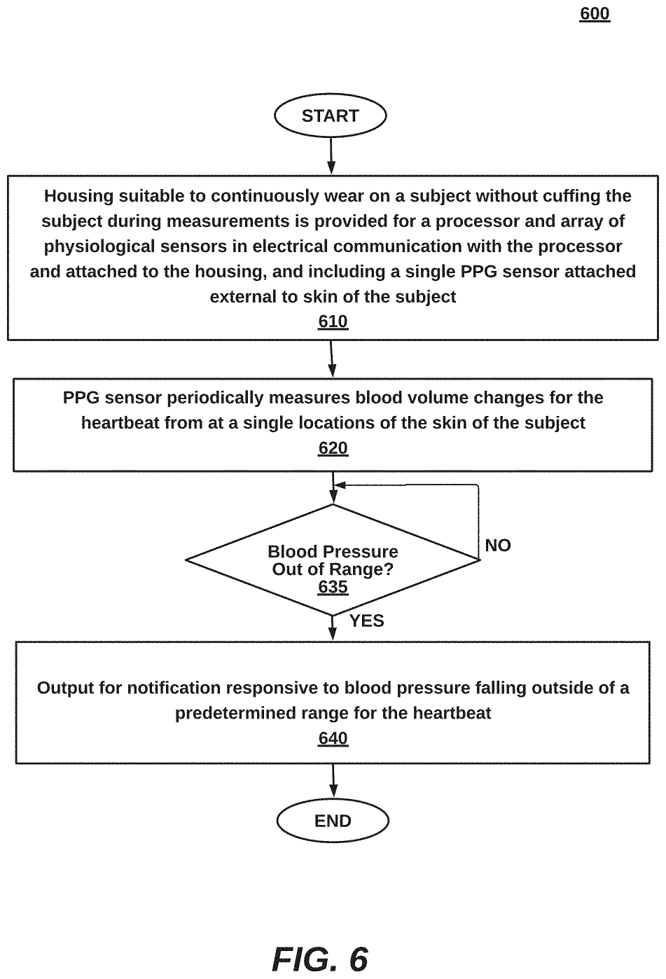

FIG. 6 is a flow chart illustrating a method 600 for wearable blood pressure monitoring device for non-invasive measurement of continuous and cuffless blood pressure readings for cardiac activity of a subject, with a single PPG sensor.

At step 610 a housing suitable to continuously wear on a subject without cuffing the subject during measurements is provided for a processor and array of physiological sensors in electrical communication with the processor and attached to the housing, and including a single PPG sensor in contact with the external surface of the skin of the subject.

At step 620, the PPG sensor periodically measures blood volume changes resulted from the heartbeat from the skin of the subject.

At step 630, the processor determines blood pressure by utilizing the temporal information of the measured PPG signal resulted from the heartbeat.

At step 635, the processor determines if the determined blood pressure is outside of a predetermined range for the heartbeat. Other embodiments perform or do not perform step 635.

At step 640, an output for notification responsive to blood pressure falling outside of the predetermined range.

In other embodiments, the blood pressure SBP/DBP is presented in real-time with or without determining if the blood pressure is out of the predetermined range (step 445, 535, 635).

Many of the functionalities described herein can be implemented with computer software, computer hardware, or any combination thereof.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the present invention.

What is claimed is:

1. A wearable blood pressure monitoring device for continuous and cuffless blood pressure readings for cardiac activity of a subject, the device comprising:
a housing suitable to continuously wear on a subject without cuffing the subject during measurements;
a processor within the housing;
an array of physiological sensors in communication with the processor, and comprising an ECG (Electrocardiogram) sensor and a PPG (Photoplethysmogram) sensor in contact with the external surface of the skin of the subject,
wherein the ECG sensor having electrodes to periodically measure electrical potential from a heartbeat from the electrodes at different locations on the skin of the subject,
wherein the PPG sensor periodically measures blood volume changes from the heartbeat from at least one location of the skin of the subject,
wherein the processor determines blood pressure from a determined PTT (Pulse Transit Time) value; and
an I/O (input/output) module for notification responsive to blood pressure.

2. The wearable blood pressure monitoring device of claim 1, wherein the processor determines blood pressure from a determined PTT value resulting from a time difference in values between the measured ECG signal at a predetermined location on an ECG waveform and the measured PPG signal at a predetermined location on a PPG waveform.

3. The wearable blood pressure monitoring device of claim 1, wherein the processor, the ECG sensor and the PPG sensor are disposed on a flexible motherboard.

4. The wearable blood pressure monitoring device of claim 1, wherein the wearable blood pressure monitoring device is implemented within at least one device from the group comprising: a ring, a wrist band/bracelet, a watch, an arm band, a necklace, a headset, an earbud, a belt, a waist band, a patch, a garment, a shoe accessory, an ankle band.

5. The wearable blood pressure monitoring device of claim 1, wherein the wearable blood pressure monitoring device is implemented within at least one wearable fabric garment from the group of comprising: a shirt, a pants, a shoe, a hat, a glove, underwear, a sock, and a band.

6. The wearable blood pressure monitoring device of claim 1, wherein at least one of electrodes for the ECG sensor is in wireless communication with the processor.

7. The wearable blood pressure monitoring device of claim 1, wherein the PPG sensor is in wireless communication with the processor.

8. The wearable blood pressure monitoring device of claim 1, wherein:
the processor estimates the PTT values based on the difference in timing of the ECG signal and PPG signals;
the processor predicts blood pressure values based on PTT values with at least one machine learning or deep learning algorithm from the group comprising: Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks.

9. The wearable blood pressure monitoring device of claim 1, wherein:
the processor estimates the blood pressure SBP/DBP (systolic blood pressure/diastolic blood pressure) values by considering ECG and PPG signals as multivariate time series using at least one machine learning or deep learning algorithm from the group comprising: Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks, LSTM.

10. The wearable blood pressure monitoring device of claim 1, wherein:
the processor converts the ECG and PPG signals into multilayer graphs using VG algorithm; estimating SBP/DBP values based on multilayer graphs with CNN.

11. A method for a wearable blood pressure monitoring device for continuous and cuffless blood pressure readings for cardiac activity of a subject, the method comprising:
attaching a processor and an array or physiological sensors in a housing suitable to continuously wear on a subject without cuffing the subject during measurements, wherein the array of physiological sensors in electrical communication with the processor and attached to the housing, and including an ECG (Electrocardiogram) sensor and a PPG (Photoplethysmogram) sensor in contact with the external surface of the skin of the subject;
periodically measuring, with the ECG sensor having electrodes, electrical potential for a heartbeat from the electrodes at different locations on the skin of the subject;
periodically measuring, with the PPG sensor, blood volume changes resulting from the heartbeat from at least one location of the skin of the subject,
determining, with the processor, blood pressure from a determined PTT (Pulse Transit Time) value resulting from a time difference in values between the measured ECG signal at a predetermined location on an ECG waveform and the measured PPG signal at a predetermined location on a PPG waveform; and
outputting for notification responsive to blood pressure falling outside of a predetermined range for the heartbeat.

12. The method of claim 11, wherein the continuous and cuffless blood pressure readings are of a human subject or a non-human subject.

13. The method of claim 11, wherein the processor, the ECG sensor and the PPG sensor are disposed on a flexible motherboard.

14. The method of claim 11, wherein the wearable blood pressure monitoring device is implemented within at least one device from the group comprising: a ring, a wrist band/bracelet, a watch, a necklace, an earbud, a belt, a waist band, and an ankle band.

15. The method of claim 11, wherein the wearable blood pressure monitoring device is implemented within at least one wearable fabric garment from the group of comprising: a shirt, a pants, a shoe, a hat, a glove, underwear, a sock, and a band.

16. The method of claim 11, wherein at least one of electrodes for the ECG sensor is in wireless communication with the processor.

17. The method of claim 11, wherein a part of the PPG sensor is in wireless communication with the processor.

18. The method of claim 11, further comprising:
estimating the PTT values based on the difference in timing of the ECG signal and PPG signals;
predicting blood pressure values based on PTT values with at least one machine learning or deep learning algorithm from the group comprising:
Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks.

19. The method of claim 11, further comprising:
estimating SBP/DBP (systolic blood pressure/diastolic blood pressure) values by considering ECG and PPG signals as multivariate time series using at least one machine learning or deep learning algorithm from the group comprising: Linear Regression, Bayesian Linear Regression, Lasso Regression, Ridge Regression, ElasticNet Regression, Multiple Regression, Multivariate Regression, Polynomial Regression, Support Vector Machine, Random Forest, k-Nearest Neighbors, Discriminant Analysis, Neural Networks, LSTM.

20. The method of claim 11, further comprising:
converting the ECG and PPG signals into multilayer graphs using VG algorithm; estimating SBP/DBP values based on multilayer graphs with CNN.
